(19) United States
(12) Patent Application Publication
(10) Pub. No.: US 2020/0170615 A1
(43) Pub. Date: Jun. 4, 2020

(54) ULTRASOUND SYSTEM WITH EXTRACTION OF IMAGE PLANES FROM VOLUME DATA USING TOUCH INTERACTION WITH AN IMAGE

(71) Applicant: KONINKLIJKE PHILIPS N.V., EINDHOVEN (NL)

(72) Inventor: David Nigel Roundhill, Woodinville, WA (US)

(73) Assignee: KONINKLIJKE PHILIPS N.V., EINDHOVEN (NL)

(21) Appl. No.: 16/639,649

(22) PCT Filed: Aug. 10, 2018
(86) PCT No.: PCT/EP2018/071721

§ 371 Date: Feb. 17, 2020

Related U.S. Application Data
(60) Provisional application No. 62/546,590, filed on Aug. 17, 2017.

(51) Int. Cl. A61B 8/08; A61B 8/13; A61B 8/00; G06T 7/00; G06T 17/10

(52) U.S. Cl. CPC A61B 8/0875; A61B 8/0866; A61B 8/13; A61B 8/465; G06T 2207/10136; G06T 7/0012; G06T 17/10; G06T 2207/30008; A61B 8/5223

(57) ABSTRACT

An ultrasound system includes an image extraction processor which is responsive to the touching of at least a portion of desired anatomy in an ultrasound image on a touchscreen display to extract an image of the desired anatomy from a 3D volumetric data set which includes the desired anatomy. The system and method can also be used to extract standard view images from volumetric image data of anatomy.

This invention relates to medical diagnostic ultrasound systems and, in particular, to ultrasound systems which enable the extraction of image planes of selected anatomy by touch interaction with an image.

Obstetrical fetal imaging is one of the most important branches of ultrasonic imaging. Ultrasound is a non-ionizing imaging modality and hence safe for developing fetuses. Ultrasound imaging is used to monitor fetal development and also to predict expected delivery dates from estimations of fetal age. Fetal age estimation is done by measuring the dimensions of various bones of a developing fetus, such as the skull and limbs. Clinically validated algorithms use these measurements in combination to estimate fetal age. A typical ultrasound system configured for obstetrical imaging is equipped with protocols to guide a sonographer in acquiring the necessary images for the measurements and also will have the age estimation algorithms onboard the system.

Acquiring the necessary images for accurate measurements of the fetal bone structure is not always easy, however. The most accurate measurements are made when the longitudinal dimension of a limb bone is fully captured in a two-dimensional (2D) image plane, but manipulating the image plane with a standard 2D imaging probe is often problematic. The fetus can move frequently and can assume various positions in the womb, putting the limbs into orientations which are not always accessible to the 2D plane extending from the probe. A solution for this situation is provided by 3D volumetric imaging. The 3D volumetric region extending from the aperture of a 3D imaging probe can be positioned to capture the volume in which the fetus is located, regardless of the current position of the fetus. A volume image can be captured quickly and then analyzed and diagnosed at leisure by viewing different slice planes of the volume using multiplanar reformatting to extract desired planes containing the necessary bones for measurement. But a problem often encountered in such extraction is that the desired anatomy may not always be clearly visible, as it can be obstructed by surrounding tissue, the umbilical cord, or the wall of the uterus. Thus it sometimes becomes necessary to “trim” away obscuring tissue in a volume image and search carefully to find the images of the tiny structures required for measurements. It is desirable to expedite this process so that the desired anatomy can be viewed quickly and completely for accurate measurement.

In accordance with the principles of the present invention, an ultrasound system enables extraction of image planes of desired anatomy from a 3D volume image dataset using image processing which has been programmed to identify the desired anatomy in 3D volume images. A 3D volume image dataset is acquired and an ultrasound image displayed on a touchscreen display. When a user sees a section of desired anatomy in an ultrasound image, the user touches the anatomy on the touchscreen. This sends a cue to an image extraction processor, which then examines image planes around the identified anatomical location and locates an image plane containing the desired anatomy. An image of the identified anatomy is displayed to a user, and the display may also automatically include a measurement of the desired anatomy useful for a particular exam such as a fetal exam.

In the drawings:

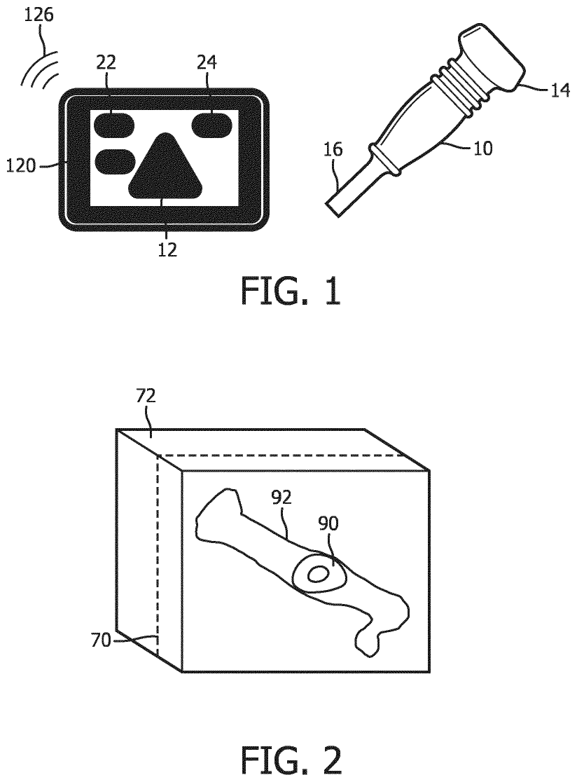

FIG. 1 illustrates a tablet ultrasound system with a touchscreen display and an ultrasound probe for use with the system.

FIG. 2 illustrates a 3D volume image containing a fetal femur and a cut plane intersecting the femur.

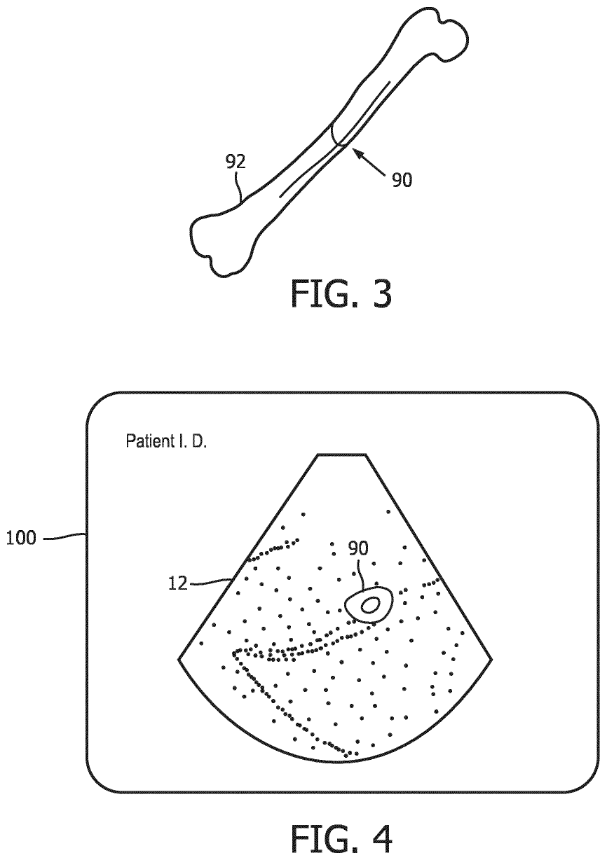

FIG. 3 is a picture of a femur indicating the location on the bone where it is intersected by the cut plane when imaged with a volume image as shown in FIG. 2.

FIG. 4 is an ultrasound image display showing an image of the cut plane of FIG. 2.

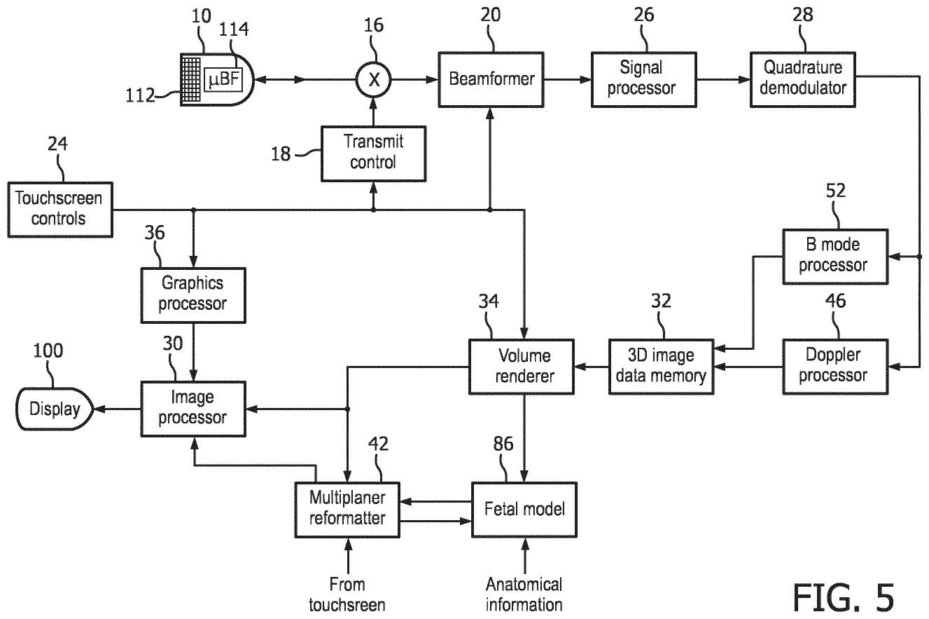

FIG. 5 is a block diagram of an ultrasound system constructed in accordance with a first implementation of the present invention which uses a fetal model to identify the image planes of fetal bones in volume image data.

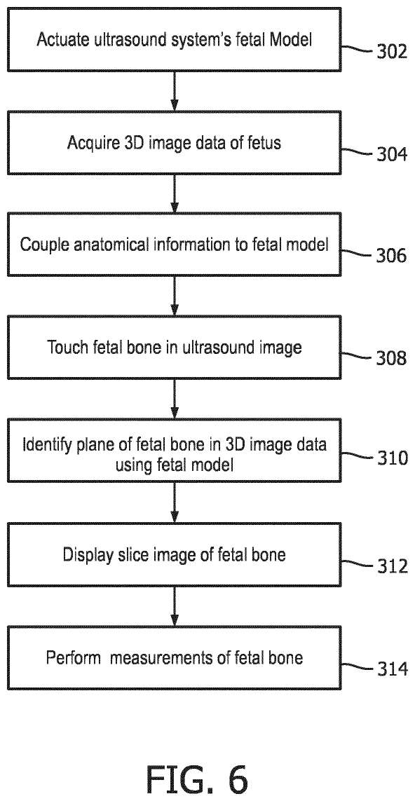

FIG. 6 is a flowchart illustrating the operation of the ultrasound system of FIG. 5.

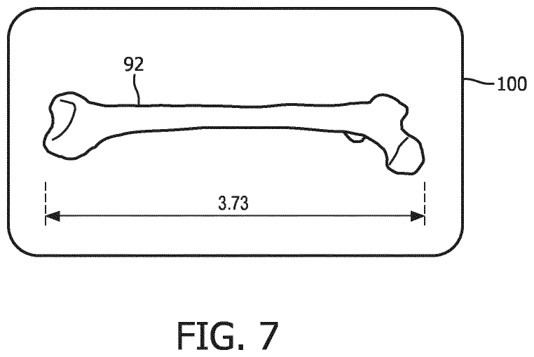

FIG. 7 is an ultrasound display of a fetal bone in a longitudinal view suitable for measurement which has been identified and extracted in accordance with the principles of the present invention.

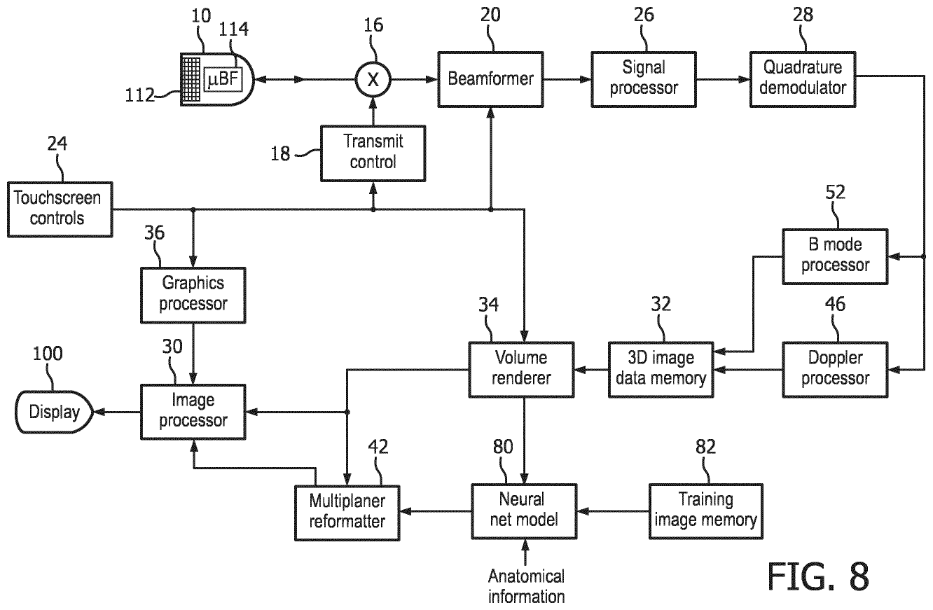

FIG. 8 is a block diagram of an ultrasound system constructed in accordance with a second implementation of the present invention which uses a neural network model to identify the image planes of fetal bones in volume image data.

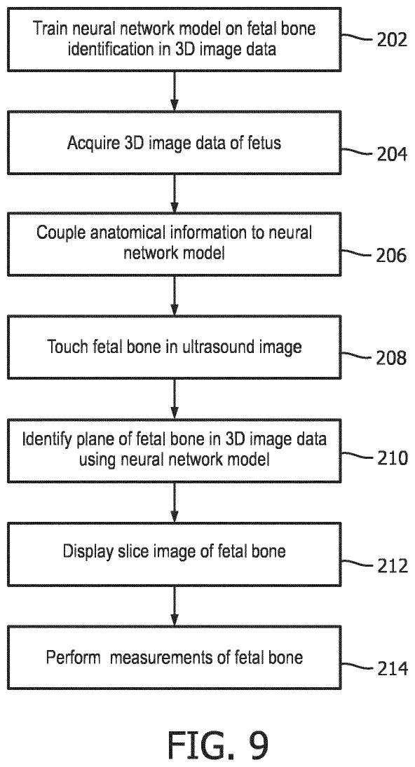

FIG. 9 is a flowchart illustrating the operation of the ultrasound system of FIG. 8.

Referring to FIG. 1, an ultrasound system of the present invention is shown. The system comprises a tablet ultrasound system with a touchscreen 120. A suitable commercial system with these characteristics is the Lumify™ ultrasound system, available from Philips Healthcare of Andover, Mass. The touchscreen display will display an ultrasound image 12 as well as patient and system information 22 and touchscreen controls 24 by which a user controls the operation of the ultrasound system. To the right of the tablet system is an ultrasound probe 10 which transmits and receives ultrasonic energy with a two-dimensional transducer array located at its distal end 14. The two-dimensional array is capable of electronically scanning and acquiring echo signals for imaging over a volumetric region of a subject. Three-dimensional imaging may also be performed with a transducer having an oscillating one-dimensional array transducer. The ultrasound probe 10 is coupled either by a cable or wirelessly to the tablet ultrasound system, which is capable of Bluetooth and Wifi communication as indicated by waves 126. The probe 10 is shown with a stub antenna 16 at its proximal end for communication with the tablet ultrasound system.

FIG. 2 depicts a volume 72 of spatially arranged image voxels produced from echoes acquired by the ultrasound probe 10 for 3D imaging. In this example the volume of image data contains image data of a fetal femur 92 which was in the scanning region of the probe. The femur is shown in dotted phantom, depicting that the bone is obscured inside the volume by surrounding voxels of tissue. The longitudinal dimension of the femur extends from the front to the back of the volume 72. A cut plane 70 through the volume of image data is also illustrated. In this example the plane 70 of pixels of image data intersects the femur 90. Thus, the image produced from the pixels in image plane 70 will include a cross-sectional area 90 of the femur bone 92. FIG. 3 is a picture of a femur bone 92 in perspective which indicates the location 90 of the slice through the bone which is in image plane 70 in FIG. 2.

With this as background, FIG. 4 shows an ultrasound system display 100 displaying a planar ultrasound image 12. This image typifies how an image of slice plane 70 will appear, including the cross-sectional view 90 of the femur 92 surrounded by pixels of the tissue located around the bone. In an implementation of the present invention, a user touches the section 90 of the femur on the display which is visible in the image. Touching the touchscreen sends a signal to the ultrasound system, indicating to the system a location in the image data around which the system is to analyze the volumetric image data to find an image plane containing a longitudinal view of the femur. The system already knows that the user is conducting an obstetrical exam, which was made known to the system when the user selected an obstetrical probe 10. Optionally, the user may also indicate to the system that it is a fetal femur bone which is to be located. An onboard image extraction processor now explores the volumetric image data in differently oriented planes which intersect the location in the image data marked by the touch of the user on bone section 90 on the touchscreen display until a plane containing a longitudinal image of the femur bone is found.

One implementation of an ultrasound system configured to perform this analysis and produce the desired image is shown in block diagram form in FIG. 5. A transducer array 112 is provided in an ultrasound probe 10 for transmitting ultrasonic waves and receiving echo information over a volumetric region of the body. The transducer array 112 may be a two-dimensional array of transducer elements capable of electronically scanning in two or three dimensions, in both elevation (in 3D) and azimuth, as shown in the drawing. Alternatively, the transducer may be a one-dimensional array capable of scanning image planes which is oscillated back and forth to sweep the image plane through a volumetric region and thereby scan the region for three-dimensional imaging, such as that described in U.S. Pat. No. 7,497,830 (Li et al.) A two-dimensional transducer array 112 is coupled to a microbeamformer 114 in the probe which controls transmission and reception of signals by the array elements. Microbeamformers are capable of at least partial beamforming of the signals received by groups or “patches” of transducer elements as described in U.S. Pat. Nos. 5,997,479 (Savord et al.), 6,013,032 (Savord), and 6,623,432 (Powers et al.) The microbeamformer is coupled by the probe cable to a transmit/receive (T/R) switch 16 which switches between transmission and reception and protects the main system beamformer 20 from high energy transmit signals. The transmission of ultrasonic beams from the transducer array 112 under control of the microbeamformer 114 is directed by a transmit controller 18 coupled to the T/R switch and the beamformer 20, which receives input from the user's operation of the user interface or controls 24 on the touchscreen display. Among the transmit characteristics controlled by the transmit controller are the spacing, amplitude, phase, and polarity of transmit waveforms. Beams formed in the direction of pulse transmission may be steered straight ahead from the transducer array, or at different angles for a wider sector field of view, the latter being typical of most obstetrical imaging probes.

The echoes received by a contiguous group of transducer elements are beamformed by appropriately delaying them and then combining them. The partially beamformed signals produced by the microbeamformer 114 from each patch are coupled to a main beamformer 20 where partially beamformed signals from individual patches of transducer elements are delayed and combined into a fully beamformed coherent echo signal. For example, the main beamformer 20 may have 128 channels, each of which receives a partially beamformed signal from a patch of 12 transducer elements. In this way the signals received by over 1500 transducer elements of a two-dimensional array transducer can contribute efficiently to a single beamformed signal.

The coherent echo signals undergo signal processing by a signal processor 26, which includes filtering by a digital filter and noise reduction as by spatial or frequency compounding. The digital filter of the signal processor 26 can be a filter of the type disclosed in U.S. Pat. No. 5,833,613 (Averkiou et al.), for example. The processed echo signals are demodulated into quadrature (I and Q) components by a quadrature demodulator 28, which provides signal phase information and can also shift the signal information to a baseband range of frequencies.

The beamformed and processed coherent echo signals are coupled to a B mode processor 52 which produces a B mode image of structure in the body such as tissue. The B mode processor performs amplitude (envelope) detection of quadrature demodulated I and Q signal components by calculating the echo signal amplitude in the form of (I²+Q²)^{½}. The quadrature echo signal components are also coupled to a Doppler processor 46, which stores ensembles of echo signals from discrete points in an image field which are then used to estimate the Doppler shift at points in the image with a fast Fourier transform (FFT) processor. The Doppler shift is proportional to motion at points in the image field, e.g., blood flow and tissue motion. For a color Doppler image, which may be formed for analysis of fetal blood flow, the estimated Doppler flow values at each point in a blood vessel are wall filtered and converted to color values using a look-up table. Either the B mode image or the Doppler image may be displayed alone, or the two shown together in anatomical registration in which the color Doppler overlay shows the blood flow in tissue and vessels in the imaged region.

The B mode image signals and the Doppler flow values when used are coupled to a 3D image data memory, which stores the image data in x, y, and z addressable memory locations corresponding to spatial locations in a scanned volumetric region of a subject. This volumetric image data is coupled to a volume renderer 34 which converts the echo signals of a 3D data set into a projected 3D image as viewed from a given reference point as described in U.S. Pat. No. 6,530,885 (Entrekin et al.) The reference point, the perspective from which the imaged volume is viewed, may be changed by a control on the touchscreen display, which enables the volume to be tilted or rotated to diagnose the region from different viewpoints.

In accordance with the principles of the present invention the volumetric image data used to produce the volume rendering is coupled to an image extraction processor which in this implementation is a fetal model 86. The fetal model is a processor and memory which stores a library of differently sized and/or shaped models in data form of typical structures of interest in a fetal exam. The library may contain different sets of models, each representing typical fetal structure at a particular age of fetal development, such as the first and second trimesters of development, for instance. The models are data representing meshes of bones of the fetal skeleton and skin (surface) of a developing fetus. The meshes of the bones are interconnected as are the actual bones of a skeleton so that their relative movements and ranges of articulation are constrained in the same manner as are those of an actual skeletal structure. Similarly the surface mesh is constrained to be within a certain range of distance of the bones it surrounds. When the user has informed the system of known anatomical information, such as the bone to be identified is believed to be a femur of a fetus in the second trimester, this information is coupled to the fetal model and used to select a particular model from the library as the starting point for analysis. The models are deformable within constraint limits, e.g., fetal age, by altering parameters of a model to warp the model, such as an adaptive mesh representing an approximate surface of a typical femur, and thereby fit the model by deformation to structural landmarks in the volumetric image data set. An adaptive mesh model is desirable because it can be warped within the limits of its mesh continuity and other constraints in an effort to fit the deformed model to structure in different image planes intersecting the identified bone location 90. This process is continued by an automated shape processor until data is found in a plane which can be fitted by the model and thus identified as the desired anatomy. The planes in the volumetric image data which are examined may be selected by the fetal model operating on the volumetric image data provided by the volume renderer 34, when the bone model is configured to do this. Alternatively, a series of differently oriented image planes intersecting the specified location 90 can be extracted from the volume data by a multiplanar reformatter 42 and provided to the fetal model 86 for analysis and fitting. The multiplanar reformatter selects echo data which are received from points in a common plane in a volumetric region of the body which can be displayed as an ultrasonic image of that plane, as described in U.S. Pat. No. 6,443,896 (Detmer). In the instant system the multiplanar reformatter is programmed to couple to the fetal model a sequence of differently oriented planes of image data which intersect the location marked by the user's touch until a plane is found with image data fitted by the model. In either case, the identification of the plane of an image with the desired anatomy is coupled back to the multiplanar reformatter for display of the desired image plane. The image extraction processor may also provide and/or seek verification of the anatomy being identified, as by displaying a message “Femur?” to the user when it appears that the user is seeking to display the femur and verification of this is desired either by the processor or as reassurance to the user. The foregoing model deformation and fitting is explained in further detail in international patent application number WO 2015/019299 (Mollus et al.) entitled “MODEL-BASED SEGMENTATION OF AN ANATOMICAL STRUCTURE.” See also international patent application number WO 2010/150156 (Peters et al.) entitled “ESTABLISHING A CONTOUR OF A STRUCTURE BASED ON IMAGE INFORMATION” and US pat. appl. pub. no. 2017/0128045 (Roundhill et al.) entitled “TRANSLATION OF ULTRASOUND ARRAY RESPONSIVE TO ANATOMICAL ORIENTATION.”

Once the orientation coordinates of an image plane containing the desired anatomy has been found, in this example a longitudinal view of a femur bone, this information is coupled to the multiplanar reformatter by the fetal bone model which selects that plane of data from the volumetric image data for display. The planar image data is coupled to an image processor 30 for scan conversion if necessary and further enhancement, buffering and temporary storage for display on an image display 40. In a preferred implementation the image processor also adds the desired measurement to the image, which is readily done as ultrasonic image data is spatially accurate. A graphics processor 36 produces a display overlay containing measurement graphics and the measurement together with the image of the desired fetal bone 92 as shown on image display 100 in FIG. 7. If desired, the ultrasound system can automatically label the identified structure as a femur, and can also be configured to automatically call up the fetal age estimation program and enter the bone measurement into the program for expedited fetal age estimation.

A method for operating the ultrasound system of FIG. 5 to extract an image of desired fetal anatomy from volumetric image data is illustrated in FIG. 6. In step 302 the fetal model of the ultrasound system is actuated. In step 304 3D (volumetric) image data of a fetus is acquired. In step 306 known anatomical information is optionally coupled to the fetal model, such as the particular bone to be identified and the age (trimester) of the fetus. In step 308 a user touches the desired fetal bone on a touchscreen display of an ultrasound image in which at least a portion of the bone is visible. This touch identification is used by a fetal model in step 310 to identify an image plane containing the desired bone in the 3D image data. In step 312 a slice image of an identified image plane of the desired fetal bone in longitudinal view is displayed. In step 314 measurements of the fetal bone are performed which, optionally, may be done automatically as described above.

FIG. 8 illustrates in block diagram form an ultrasound system comprising a second implementation of the present invention. In the system of FIG. 8, system elements which were shown and described in FIG. 5 are used for like functions and operations and will not be described again. In the system of FIG. 8 the image extraction processor comprises a neural net model 80. A neural net model makes use of a development in artificial intelligence known as “deep learning.” Deep learning is a rapidly developing branch of machine learning algorithms that mimic the functioning of the human brain in analyzing problems. The human brain recalls what was learned from solving a similar problem in the past, and applied that knowledge to solve a new problem. Exploration is underway to ascertain possible uses of this technology in a number of areas such as pattern recognition, natural language processing and computer vision. Deep learning algorithms have a distinct advantage over traditional forms of computer programming algorithms in that they can be generalized and trained to recognize image features by analyzing image samples rather than writing custom computer code. The anatomy visualized in an ultrasound system would not seem to readily lend itself to automated image recognition, however. Every person is different, and anatomical shapes, sizes, positions and functionality vary from person to person. Furthermore, the quality and clarity of ultrasound images will vary even when using the same ultrasound system. That is because body habitus will affect the ultrasound signals returned from the interior of the body which are used to form the images. Scanning a fetus through the abdomen of an expectant mother will often result in greatly attenuated ultrasound signals and poorly defined anatomy in the fetal images. Nevertheless, the system described in this application has demonstrated the ability to use deep learning technology to recognize anatomy in fetal ultrasound images through processing by a neural network model. The neural network model is first trained by presenting to it a plurality of images of known anatomy, such as fetal images with known fetal structure which is identified to the model. Once trained, live images acquired by a user during a fetal exam are analyzed by the neural net model in real time, which identifies the anatomy in the images.

Deep learning neural net models comprise software which may be written by a software designer, and are also publicly available from a number of sources. In the ultrasound system of FIG. 8, the neural net model software is stored in a digital memory. An application which can be used to build a neural net model called “NVidia Digits” is available at https://developer.nvidia.com/digits. NVidia Digits is a high-level user interface around a deep learning framework called “Caffe” which has been developed by the Berkley Vision and Learning Center, http://caffe.berkeleyvision.org/. A list of common deep learning frameworks suitable for use in an implementation of the present invention is found at https://developer.nvidia.com/deep-learning-frameworks. Coupled to the neural net model 80 is a training image memory 82, in which ultrasound images of known fetal anatomy including fetal bones are stored and used to train the neural net model to identify that anatomy in 3D (volumetric) ultrasound image data sets. Once the neural net model is trained by a large number of known fetal images, the neural net model receives a volume image data set of a fetus from the volume renderer 34. The neural net model may receive other cues in the form of anatomical information such as the fact that an obstetrical exam is being performed and the trimester of the fetus, as described above. The neural net model also receives the locational signal generated by a user touching a portion of the desired anatomy on a touchscreen display, a femur bone in this example. The neural net model then analyzes regions including the identified location until a femur bone is identified in the volume image data. The coordinates of a plane containing longitudinal image data of the femur are coupled to the multiplanar reformatter 42, which extracts the desired femur image from the volumetric image data set and forwards it to the image processor for display as shown in FIG. 7. As before, the ultrasound system may be conditioned to automatically label and/or measure the bone, display the measurement, and couple the measurement information to another program such as a gestational age estimation program.

A method for operating the ultrasound system of FIG. 8 to extract an image of desired fetal anatomy from volumetric image data is illustrated in FIG. 9. In step 202 a neural network model is trained to identify fetal bones in 3D fetal image data. In step 204 3D (volumetric) image data of a fetus is acquired. In step 206 known anatomical information is optionally coupled to the fetal bone model, such as the particular bone to be identified and the age (trimester) of the fetus. In step 208 a user touches the desired fetal bone on a touchscreen display of an ultrasound image in which at least a portion of the bone is visible. This touch identification of location in an image is used by a neural network model in step 210 to identify an image plane containing the desired bone in the 3D image data. In step 212 a slice image of an identified image plane of the desired fetal bone is displayed. In step 214 measurements of the fetal bone are performed which, optionally, may be done automatically as described above.

Variations of the systems and methods described above will readily occur to those skilled in the art. A number of system components shown in FIGS. 5 and 8 can be located in the probe case. Some Lumify probes, for example, contain components from the transducer through the B mode processor, outputting to the tablet display detected image signals over a USB cable. This methodology can be extended to include even additional components in the probe, if desired, such as the 3D image data memory and volume rendering software. Thus, a number of components which are described above as “system” components may alternatively be located in the ultrasound probe.

The techniques of the present invention can be used in other diagnostic areas besides obstetrics. For instance, numerous ultrasound exams require standard views of anatomy for diagnosis. In diagnoses of the kidney, a standard view is a coronal image plane of the kidney. In cardiology, two-chamber, three-chamber, and four-chamber views of the heart are standard views. A neural network model can be trained to recognize such views in 3D image data sets of the heart and then be used to select image planes of desired views from volumetric data and display them to a clinician. Other applications will readily occur to those skilled in the art.

It should be noted that an ultrasound system suitable for use in an implementation of the present invention, and in particular the component structure of the ultrasound systems of FIGS. 5 and 8, may be implemented in hardware, software or a combination thereof. The various embodiments and/or components of an ultrasound system, for example, the fetal bone model and deep learning software modules, or components, processors, and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system or the data network for importing training images. The computer or processor may also include a memory. The memory devices such as the 3D image data memory 32, the training image memory, and the memory storing fetal bone model libraries may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system including those controlling the acquisition, processing, and transmission of ultrasound images as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules such as a neural network model module, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasonic diagnostic imaging system for extracting a desired view of anatomy from volume image data which includes the anatomy comprising:
an ultrasound probe adapted to acquire volume image data which includes image data of a desired anatomy;
a display adapted to display an ultrasound image from the acquired image data showing at least a portion of the desired anatomy on a touchscreen display;
an image extraction processor, responsive to the volume image data and a touch of the desired anatomy on the touchscreen display, the touch corresponding to a touch location, and adapted to locate from the volume image data an image plane consisting the desired anatomy indicated by the touch of the user, based on examining image planes from the volume image data which intersect the location the user's touch,
and extract said identified image plane of the desired anatomy from the volume image data;
wherein the display is further adapted to display the extracted image plane of the desired anatomy.

2. The ultrasonic diagnostic imaging system of claim 1, further comprising a B mode processor,
wherein the image extraction processor further comprises a fetal model.

3. The ultrasonic diagnostic imaging system of claim 1, further comprising a B mode processor,
wherein the image extraction processor further comprises a neural network model.

4. The ultrasonic diagnostic imaging system of claim 3, wherein the neural network model is adapted to be trained with known images of the desired anatomy.

5. The ultrasonic diagnostic imaging system of claim 4, wherein the neural network model is further adapted to recognize the desired anatomy in B mode image data of the volume image data.

6. The ultrasonic diagnostic imaging system of claim 5, wherein the neural network model is further adapted to recognize a plane of image data containing the desired anatomy in B mode volume image data.

7. The ultrasonic diagnostic imaging system of claim 6, further comprising a multiplanar reformatter, responsive to the volume image data and the recognition of a plane of image data containing the desired anatomy by the neural network model, which is adapted to produce an image plane of image data containing the desired anatomy from the volume image data.

8. The ultrasonic diagnostic imaging system of claim 3, wherein the system is further responsive to the extraction of an image of desired anatomy by the neural network model and adapted to produce a measurement of the desired anatomy.

9. The ultrasonic diagnostic imaging system of claim 3, wherein the desired anatomy is a fetal bone;
wherein the image of the desired anatomy is an image of the fetal bone; and
wherein the display is further adapted to display a measurement of the fetal bone.

10. The ultrasonic diagnostic imaging system of claim 9, wherein the system is further adapted to use the measurement of the fetal bone in a fetal age estimation.

11. The ultrasonic diagnostic imaging system of claim 1, wherein the extracted image further comprises a standard view of the desired anatomy.

12. A method of producing a desired view of desired anatomy during ultrasound imaging comprising:
acquiring volume image data which includes image data of the desired anatomy;
displaying an image from the volume image data which includes at least a portion of the desired anatomy on a touchscreen display;
sensing a user touch on the at least a portion of the desired anatomy on the touchscreen display, the touch having a touch location;
in response to the touching, locating from the volume image data an image plane containing the desired anatomy indicated by the touch of the user, based on examining image planes from the volume image data which intersect the location of the user's touch, and extracting the identified image plane of the desired anatomy from the volume image data; and
displaying the extracted image plane of the desired anatomy.

13. The method of claim 12, wherein the extracting is performed using a neural net model.

14. The method of claim 12, wherein the extracting is performed using a fetal model.

15. (canceled)

16. The method of claim 12, wherein the desired image further comprises a standard view of the desired anatomy.
